(19) United States
(12) Patent Application Publication
(10) Pub. No.: US 2019/0059791 A1
(43) Pub. Date: Feb. 28, 2019

(54) MOTION ESTIMATION SYSTEM, MOTION ESTIMATION METHOD, AND WEARABLE DEVICE

(71) Applicants: DENSO CORPORATION, Kariya-city, Aichi-pref. (JP); ISUZU MOTORS LIMITED, Tokyo (JP)

(72) Inventors: Tetsushi NORO, Kariya-city (JP); Shinji NIWA, Kariya-city (JP); Tadashi KAMADA, Kariya-city (JP); Yuuki MORI, Kariya-city (JP); Hirokazu OOYABU, Kariya-city (JP); Mayumi IWAO, Yokohama-shi (JP)

(73) Assignees: DENSO CORPORATION, Kariya-city, Aichi-pref. (JP); ISUZU MOTORS LIMITED, Tokyo (JP)

(21) Appl. No.: 15/768,961

(22) PCT Filed: Sep. 6, 2016
(86) PCT No.: PCT/JP2016/076081

§ 371 Date: Apr. 17, 2018

(30) Foreign Application Priority Data
Oct. 19, 2015 (JP) 2015-205793

(51) Int. Cl. A61B 5/11; A61B 5/00; A61B 5/05

(52) U.S. Cl. CPC A61B 5/1121; A61B 5/6803; A61B 5/6893; A61B 5/05; A61B 2560/0252; A61B 5/1116; A61B 2562/0219; A61B 2560/0214; A61B 5/002

(57) ABSTRACT

A motion estimation system includes a head motion detection unit, a mobile body motion detection unit, and a movement estimation unit. The head motion detection unit is worn on an occupant in a mobile body, and detects a motion of a head of the occupant. The mobile body motion detection unit detects a motion of the mobile body. The movement estimation unit obtains the motion of the head of the occupant and the motion of the mobile body, and estimates a movement of the head of the occupant made by the occupant according to a difference between the motion of the head of the occupant and the motion of the mobile body.

### CROSS REFERENCE TO RELATED APPLICATION

This application is based on Japanese Patent Application No. 2015-205793 filed on Oct. 19, 2015, the disclosure of which is incorporated herein by reference.

### TECHNICAL FIELD

The present disclosure relates to a motion estimation system, a motion estimation method, and a wearable device each of which detects head motion of an occupant in a mobile body, such as a vehicle.

### BACKGROUND ART

Recently, a need is increasing for a technique of detecting an orientation of a head of a driver with an aim of determining, for example, whether the driver is looking aside. Patent Literature 1 discloses one type of such a technique, according to which an image of a face of a driver is captured by a camera by irradiating illumination light toward the face from a light emitting diode, a laser light source, or the like, and an orientation of a head of the driver is detected from the captured image.

### PRIOR ART LITERATURE

### Patent Literature

Patent Literature 1: JP 2010-164393 A

### SUMMARY OF INVENTION

It is, however, anticipated that a technique of detecting an orientation of the head of the driver from an image captured by a camera as in Patent Literature 1 has a difficulty in capturing an image of the face in some circumstances, to be more specific, in a circumstance where a physical size of the driver is too small or large that the face of the driver is out of an imaging range of the camera or an arm of the driver operating a steering wheel blocks the face.

As has been described, the technique of detecting an orientation of the head by a camera raises a concern that a highly accurate detection becomes difficult depending on a location of the camera. Further, a camera capable of realizing a highly accurate detection is expensive. Hence, a need for a technique of detecting an orientation of the head of the driver without using a camera image is growing in order to constitute a system capable of measuring an orientation of the head easily or in order to complement a system using a camera.

For example, an orientation of the head may be computed by detecting motion of the head using a sensor attached to the head. However, because the driver wearing the sensor on the head is on board, motion of the head detected by the sensor inevitably includes a motion component caused by a motion of the vehicle. It is therefore difficult to correctly obtain a movement of the head made by the driver by detecting motion of the head while the sensor is attached to the driver.

In view of the foregoing difficulties, it is an object of the present disclosure to provide a motion estimation system, a motion estimation method, and a wearable device, each capable of detecting a movement of a head made by an occupant, such as a driver, with high accuracy even in a circumstance where an occupant is travelling on a mobile body, such as a vehicle.

According to an aspect of the present disclosure, a motion estimation system includes a head motion detection unit, a mobile body motion detection unit, and a movement estimation unit. The head motion detection unit is worn on an occupant in a mobile body, and detects a motion of a head of the occupant. The mobile body motion detection unit detects a motion of the mobile body. The movement estimation unit obtains the motion of the head of the occupant and the motion of the mobile body, and estimates a movement of the head of the occupant made by the occupant according to a difference between the motion of the head of the occupant and the motion of the mobile body.

According to another aspect of the present disclosure, a motion estimation method executed by at least one processor includes: a head motion obtaining step of obtaining a motion of a head of an occupant in a mobile body, the motion of the head of the occupant being detected by a wearable device worn on the occupant; a mobile body motion obtaining step of obtaining a motion of the mobile body, the motion of the mobile body being detected by a vehicle-mounted device mounted on the mobile body; and a movement estimating step of estimating a movement of the head made by the occupant according to a difference between the motion of the head of the occupant and the motion of the mobile body.

According to another aspect of the present disclosure, a wearable device employed in a motion estimation system includes a head motion detection unit detecting a motion of a head of an occupant in a mobile body. The motion estimation system includes a mobile body motion detection unit detecting a motion of the mobile body, and a movement estimation unit obtaining the motion of the head of the occupant and the motion of the mobile body. The movement estimation unit estimates a movement of the head made by the occupant according to a difference between the motion of the head and the motion of the mobile body. The wearable device is worn on the occupant.

According to the motion estimation system, the motion estimation method, and the wearable device as above, the movement estimation unit or the movement estimating step removes a component caused by motion of the mobile body from motion of the head detected at the head of the occupant according to a difference between motion of the head obtained by the head motion detection unit and motion of the mobile body obtained by the mobile body motion detection unit. Hence, a movement of the head made by the occupant, that is, a relative movement of the head with respect to the mobile body is extracted from information representing motion of the head. Consequently, a movement of the head made by the occupant can be detected with high accuracy even in a circumstance where the occupant wearing the head motion detection unit is traveling on the mobile body.

### BRIEF DESCRIPTION OF DRAWINGS

The above and other objects, features and advantages of the present invention will become more apparent from the following detailed description made with reference to the accompanying drawings. In the drawings:

FIG. 1 is a diagram schematically showing an overall motion estimation system;

FIG. 2 is a diagram of a vehicle equipped with the motion estimation system;

FIG. 3 is a block diagram showing an overall configuration of a motion estimation system according to a first embodiment;

FIG. 4 is a diagram showing a wearable device according to the first embodiment;

FIG. 5 is a flowchart showing a face orientation computation process performed by a terminal control unit in a portable terminal;

FIG. 6 is a diagram showing a change in head motion detected by a head motion detection unit;

FIG. 7 is a diagram showing a change in vehicle motion detected by a mobile body motion detection unit;

FIG. 8 is a diagram showing a change in face orientation estimated by subtracting vehicle motion from head motion;

FIG. 9 is a diagram used to describe a measurement condition of data shown in FIG. 6;

FIG. 10 is a block diagram showing an overall configuration of a motion estimation system according to a second embodiment;

FIG. 11 is a diagram showing a wearable device according to the second embodiment;

FIG. 12 is a block diagram showing an overall configuration of a motion estimation system according to a third embodiment; and

FIG. 13 is a block diagram showing an overall configuration of a motion estimation system according to a fourth embodiment.

### EMBODIMENTS FOR CARRYING OUT INVENTION

Hereinafter, several embodiments of the present disclosure will be described according to the drawings. Like components in respective embodiments below may be labelled with same reference numerals and a description may not be repeated where appropriate. When only a part of configurations is described in the respective embodiments below, a description of configurations in any preceding embodiment applies to a rest of the configurations. Besides a combination of components explicitly described in the respective embodiments below, components of two or more embodiments may be combined partially unless a trouble arises even when such a combination is not described explicitly. It is understood that a combination of components in the several embodiments and modifications below is within the scope of the disclosure described below even when such a combination is not described explicitly.

### First Embodiment

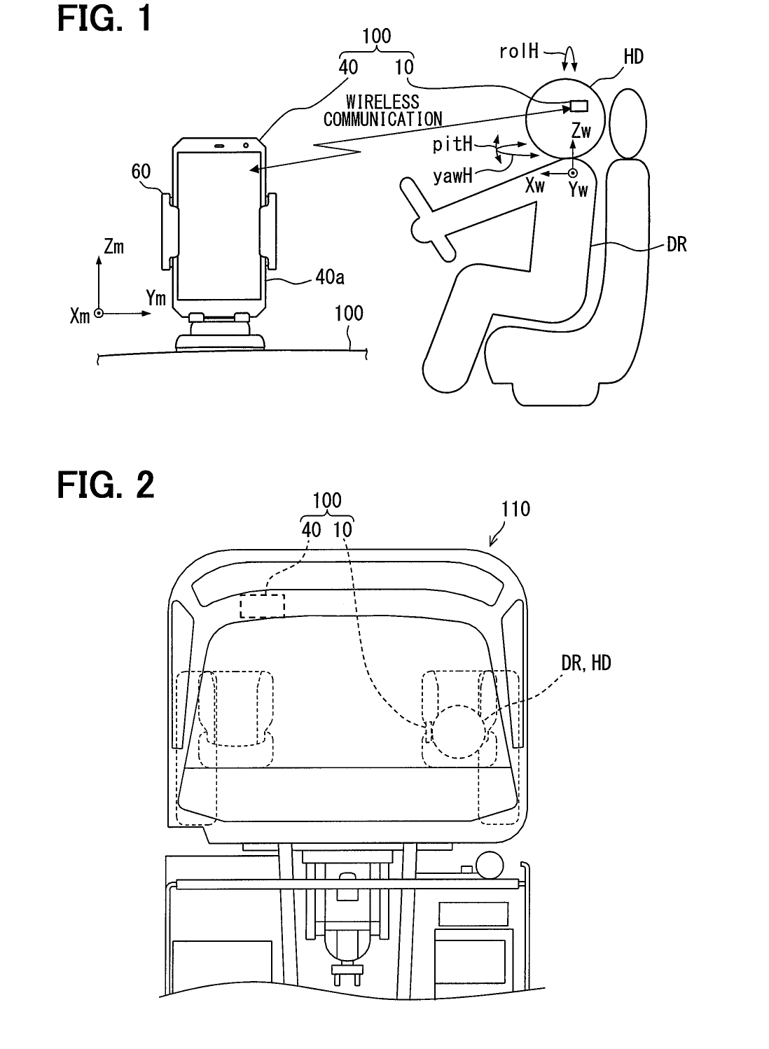

As shown in FIG. 1 through FIG. 3, a motion estimation system 100 to which the present disclosure is applied includes a wearable device 10 and a vehicle-mounted device 40 configured to make communication with each other. The motion estimation system 100 mainly functions in a compartment of a vehicle 110 as a mobile body. The motion estimation system 100 detects motion of a head HD of a driver DR in the vehicle 110 using the wearable device 10. The motion estimation system 100 computes a face orientation of the driver DR from the detected motion of the head HD.

Information on a face orientation of the driver DR acquired by the motion estimation system 100 is used in an application which determines a quality degradation of a safety confirming action, an abnormal driving state, an abnormal health state (so-called dead man), and so on. When an abnormality of the driver DR as above is detected, a warning or the like is given to the driver DR from the vehicle-mounted device 40 or any other appropriate vehicle-mounted device.

More specifically, a quality degradation of a safety confirming action is estimated by analyzing how many times, how long, and in what pattern the driver DR looks at a particular portion, such as a mirror and a meter, according to a tracking result of a face orientation. An abnormal driving state is estimated by a face orientation state, such as a state of the driver DR looking aside for a considerable time or looking down and operating a smartphone. An abnormal health state caused by a sudden death or a critical health condition of the driver DR is estimated when the posture of driver DR collapses.

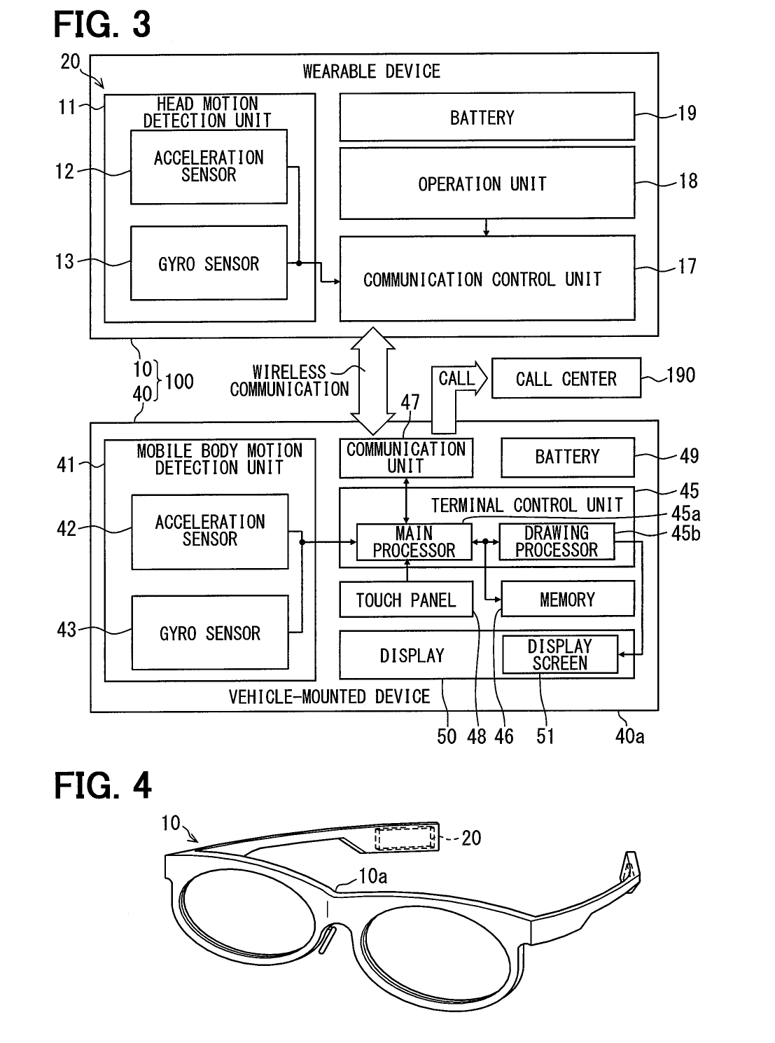

As shown in FIG. 4, the wearable device 10 is an eyeglasses-type motion sensor device, and includes a detection circuit 20 attached to eyeglasses 10a. The wearable device 10 is worn on the head HD of the driver DR as shown in FIG. 1 and successively transmits detected motion of the head HD to the vehicle-mounted device 40. As shown in FIG. 1 and FIG. 3, the detection circuit 20 in the wearable device 10 includes a head motion detection unit 11, a communication control unit 17, an operation unit 18, a battery 19, and so on.

The head motion detection unit 11 is a motion sensor detecting motion of the head HD of the driver DR wearing the wearable device 10. The head motion detection unit 11 measures acceleration, an angular velocity, and so on induced by a movement of the head HD made by the driver DR, such as an action to move the head HD in a longitudinal (pitch) direction pitH, an action to turn the head HD in a lateral (yaw) direction yawH, and an action to tilt the head HD in a right-left (roll) direction rolH. The head motion detection unit 11 has an acceleration sensor 12 and a gyro sensor 13. The head motion detection unit 11 is connected to the communication control unit 17 and outputs measurement data of the respective sensors 12 and 13 to the communication control unit 17.

The acceleration sensor 12 is configured to detect acceleration as a voltage value. The acceleration sensor 12 is capable of measuring magnitude of acceleration along each of three axes defined in the head motion detection unit 11, namely, an Xw axis, a Yw axis, and a Zw axis orthogonal to one another. The acceleration sensor 12 outputs acceleration data of the respective three axes to the communication control unit 17.

The gyro sensor 13 is configured to detect an angular velocity as a voltage value. The gyro sensor 13 is capable of measuring magnitude of an angular velocity induced about each one of the Xw axis, the Yw axis, and the Zw axis. The gyro sensor 13 measures magnitude of an angular velocity induced by a movement of the head HD made by the driver DR, and outputs angular velocity data of the respective three axes to the communication control unit 17.

The Xw axis, the Yw axis, and the Zw axis defined in the respective sensors 12 and 13 do not necessarily coincide with respective virtual rotation axes in the directions pitH, yawH, and rolH relating to head movements, and may be displaced from the respective virtual rotation axes.

The communication control unit 17 is capable of transmitting information to and receiving information from the vehicle-mounted device 40 by wireless communication, for example, Bluetooth (registered trademark) or a wireless LAN. The communication control unit 17 has an antenna in compliance with wireless communication standards. The communication control unit 17 is electrically connected to the acceleration sensor 12 and the gyro sensor 13 and acquires measurement data outputted from the respective sensors 12 and 13. When a connection by wireless communication is established between the communication control unit 17 and the vehicle-mounted device 40, the communication control unit 17 successively encodes the input measurement data and transmits the encoded data to the vehicle-mounted device 40.

The operation unit 18 has a power-supply switch or the like switching ON and OFF a power supply of the wearable device 10. The battery 19 is a power source for supplying the head motion detection unit 11, the communication control unit 17, and so on with operating power. The battery 19 may be a primary cell, such as lithium cell, or a secondary cell, such as a lithium-ion cell.

The vehicle-mounted device 40 is provided by a portable terminal 40a that can be brought into the vehicle 110 by the driver DR or any other individual. The portable terminal 40a is an electronic device provided with a highly sophisticated processing circuit represented by, for example, a multi-functional cell phone (so-called smartphone) or a tablet terminal. The vehicle-mounted device 40 is detachably attached to an instrument panel or the like of the vehicle 110 with a holder 60 or the like and is therefore restricted from moving relatively with respect to the vehicle 110 during driving. The vehicle-mounted device 40 includes a mobile body motion detection unit 41, a memory 46, a communication unit 47, a touch panel 48, a battery 49, a display 50, and a terminal control unit 45.

The mobile body motion detection unit 41 is a motion sensor used to detect a posture or the like of the portable terminal 40a. Once the portable terminal 40a is held by the holder 60, the mobile body motion detection unit 41 is attached to the vehicle 110 and functions as a sensor detecting motion of the vehicle 110. The mobile body motion detection unit 41 has an acceleration sensor 42 and a gyro sensor 43 operating substantially in a same manner, respectively, as the sensors 12 and 13 in the head motion detection unit 11. The respective sensors 42 and 43 are electrically connected to the terminal control unit 45.

The acceleration sensor 42 measures acceleration induced at the vehicle 110 in response to an operation by the driver DR, such as acceleration, deceleration, and steering. The acceleration sensor 42 outputs acceleration data of respective three axes defined in the mobile body motion detection unit 41, namely an Xm axis, a Ym axis, and a Zm axis, to the terminal control unit 45. The three axes defined in the mobile body motion detection unit 41 may be displaced from the three axes defined in the head motion detection unit 11.

The gyro sensor 43 measures an angular velocity induced at the vehicle 110 due to a change in posture of the vehicle 110 in response to an operation of the driver DR or any other individual. The gyro sensor 43 outputs angular velocity data of the respective three axes to the terminal control unit 45.

The memory 46 stores programs of applications and the like necessary for the portable terminal 40a to operate. To be more specific, the memory 46 is a non-transitory tangible storage medium, such as a flash memory. The memory 46 may be an internal memory of the portable terminal 40a or an external memory, such as a memory card inserted into a card slot of the portable terminal 40a. Data in the memory 46 can be read out and rewritten by the terminal control unit 45 when the memory 46 is electrically connected to the terminal control unit 45.

The communication unit 47 transmits information to and receives information from the wearable device 10 by wireless communication. The communication unit 47 is also capable of making mobile communication with a base station outside the vehicle 110. The communication unit 47 has antennae in compliance with standards of wireless communication of the respective types. The communication unit 47 successively acquires the measurement data of the acceleration sensor 12 and the gyro sensor 13 by decoding a wireless signal received from the communication control unit 17. The communication unit 47 outputs the measurement data thus acquired to the terminal control unit 45. In addition, the communication unit 47 is capable of making an emergency call to a call center 190 outside the vehicle 110 by mobile communication in the event of an abnormality of the driver DR, the vehicle 110, or the like.

The touch panel 48 is integrated with a display screen 51 of a display 50. The touch panel 48 detects an operation inputted via the display screen 51 by the driver DR or any other individual. The touch panel 48 is connected to the terminal control unit 45 and outputs an operation signal according to an input operation made by the driver DR or any other individual to the terminal control unit 45.

The battery 49 is a secondary cell, such as a lithium-ion cell. The battery 49 is a power supply of the portable terminal 40a and supplies power to the mobile body motion detection unit 41, the terminal control unit 45, the communication unit 47, the display 50, and so on.

The display 50 is a dot-matrix display instrument capable of displaying various full-color images with multiple pixels arrayed on the display screen 51. The display 50 is connected to the terminal control unit 45 and a display on the display screen 51 is controlled by the terminal control unit 45. While the portable terminal 40a is attached to the vehicle 110 by the holder 60, the display 50 is visible to the driver DR. When an application of computation process described below starts, the display 50 displays, for example, information on states of charge of the portable terminal 40a and the wearable device 10 and information on sensitivity of wireless communication.

The terminal control unit 45 is mainly formed of a microcomputer having a main processor 45a, a drawing processor 45b, a RAM, an input-output interface, and so on. The terminal control unit 45 controls the mobile body motion detection unit 41, the communication unit 47, the display 50, and so on by executing various programs stored in the memory 46 on the main processor 45a and the drawing processor 45b.

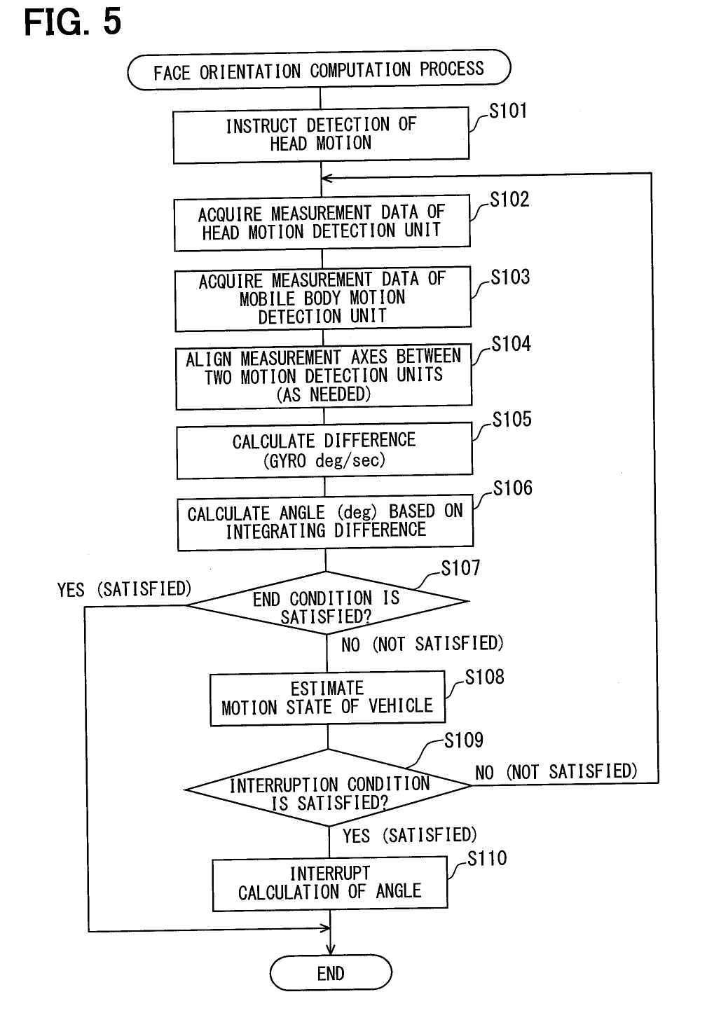

More specifically, the terminal control unit 45 is capable of computing a face orientation of the driver DR by executing a program read out from the memory 46. Face orientation computation process will be described in detail according to FIG. 5 with reference to FIG. 3 and FIG. 1. Process depicted by a flowchart of FIG. 5 is started by the terminal control unit 45 when an application to compute a face orientation is started in response to, for example, an input of an operation into the portable terminal 40a.

In S101, a command signal instructing the wearable device to start a detection of head motion is outputted to the wearable device 10. Then, advancement is made to S102. The wearable device 10 starts a detection of head motion by the head motion detection unit 11 and a transmission of measurement data by the communication control unit 17 in response to the command signal outputted from the portable terminal 40a in S101.

After a transmission of the measurement data of the head motion detection unit 11 from the wearable device 10 is started, a reception of the measurement data is started in S102. The communication unit 47, which receives the measurement data, outputs the received measurement data to the terminal control unit 45. After the terminal control unit 45 acquires the measurement data on head motion in the manner as above, advancement is made to S103. In S103, the terminal control unit acquires measurement data on vehicle motion from the mobile body motion detection unit 41. Then, advancement is made to S104.

In S104, the Xw axis, the Yw axis, and the Zw axis defined in the head motion detection unit 11 are aligned, respectively, with the Xm axis, the Ym axis, and the Zm axis defined in the mobile body motion detection unit 41. Then, advancement is made to S105. Axial alignment in S104 is performed in reference to, for example, an acting direction of gravitational acceleration detectable by the respective acceleration sensors 12 and 42.

Axial alignment in S104 may be performed as needed during the face orientation computation process. For example, axial alignment may be performed repetitively at regular time intervals or when a change in wearing posture of the wearable device 10 with respect to the head HD is estimated according to the measurement data of the head motion detection unit 11. Owing to the process in S104 as above, axial displacement between the two motion detection units 11 and 41 can be corrected whenever necessity arises, such as when the driver DR relaxes a posture consciously, when the driver DR removes and wears the wearable device 10 again for some reason, and when the wearable device 10 accidentally slips off.

In S105, angular velocities (deg/sec) of head motion about the pitch direction pitH, the yaw direction yawH, and the roll direction rolH are obtained as the measurement data of the head motion detection unit 11. Also, angular velocities of vehicle motion about the pitch direction pitH, the yaw direction yawH, and the roll direction rolH are obtained as the measurement data of the mobile body motion detection unit 41.

Differences of the angular velocities between the head motion and the vehicle motion are calculated to estimate a head movement made by the driver DR. Then, advancement is made to S106. In S106, angles of the head HD in the respective rotation directions are calculated by integrating the differences of the angular velocities calculated in S105 over time. Then, advancement is made to S107. A present face orientation of the driver DR is obtained in S107.

In S107, a determination is made as to whether an end condition of the computation process is satisfied. The end condition is satisfied when an operation to end the application is inputted, when the power supply of the vehicle 110 is turned OFF, and so on. When it is determined in S107 that the end condition is satisfied, the terminal control unit ends the face orientation computation process. Meanwhile, when it is determined in S107 that the end condition is not satisfied, advancement is made to S108.

In S108, a present motion state of the vehicle 110 is estimated according to most recently acquired measurement data of the mobile body motion detection unit 41, to be more specific, the measurement data of the acceleration sensor 42. Then, advancement is made to S109. In S108, a motion state of the vehicle 110 may be estimated by using the measurement data of the acceleration sensor 12 in the head motion detection unit 11. Alternatively, the communication unit 47 may be configured to make wireless communication with an intra-vehicle network. When configured in such a manner, the terminal control unit 45 is capable of estimating a motion state of the vehicle 110 by acquiring vehicle speed information of the vehicle 110 via the communication unit 47.

In S109, a determination is made as to whether a motion state of the vehicle 110 estimated in S108 satisfies a preliminarily set interruption condition. The interruption condition is satisfied when a motion state of the vehicle 110 indicates that the vehicle 110 is not moving, moving slowly at or below a predetermined speed (slowing down), or moving backward. When it is determined in S109 that the interruption condition is satisfied, advancement is made to S110.

In S110, computation to estimate a movement of the head HD and to calculate an angle of the head HD in S105 and S106, respectively, is temporarily interrupted and the computation process currently taking place is ended. Consumption of the battery 49 is reduced by interrupting the computation process. After the computation process is interrupted in S110, the face orientation computation process is resumed in a manual manner when an operation is made by the driver DR manually on the touch panel 48. After the computation process is interrupted in S110, the face orientation computation process may also be resumed in an automatic manner in response to an increase of the vehicle speed.

Meanwhile, when it is determined in S109 that the interruption condition is not satisfied, the flow returns to S102 to continue the computation to estimate a movement of the head HD and to calculate an angle of the head HD. By repetitively performing S102 through S106 as described above, an angle of the head HD is constantly updated to a latest value.

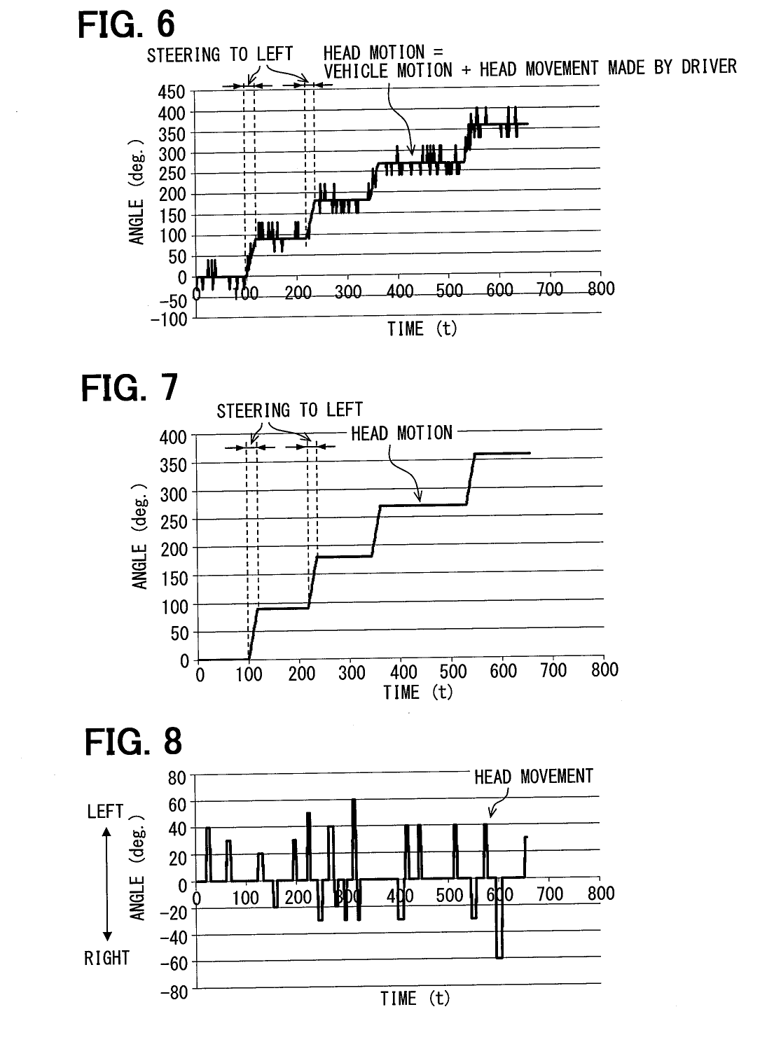

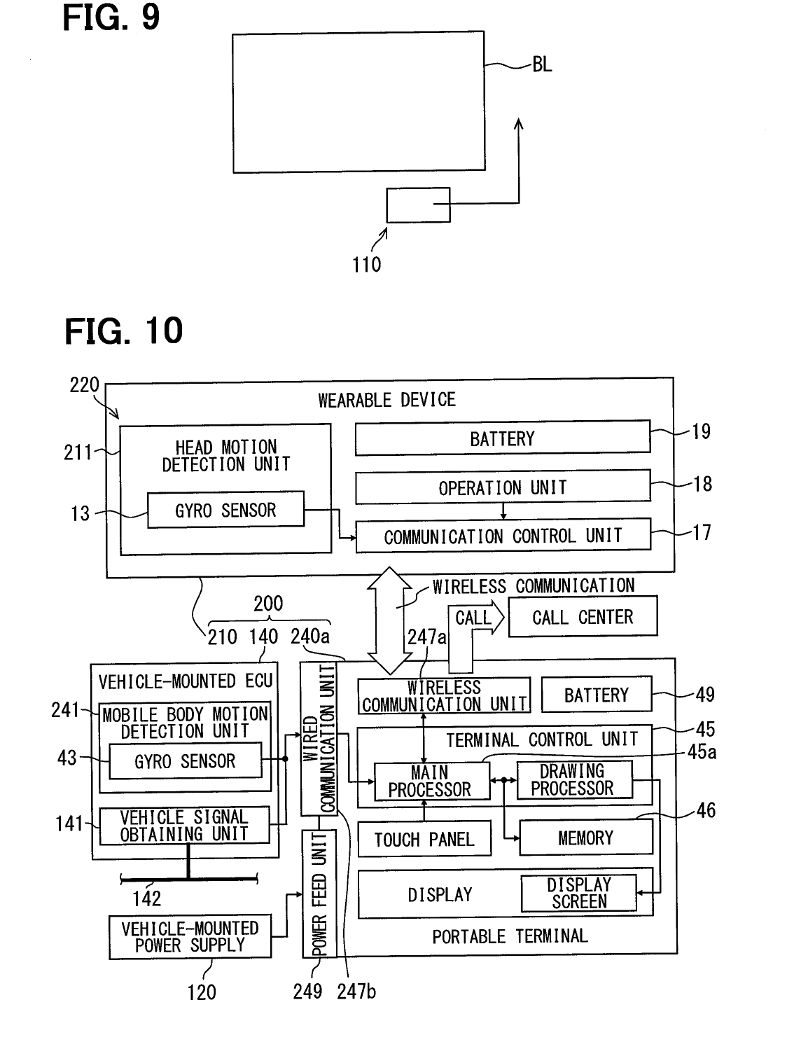

According to the computation process above, a movement of the head HD made by the driver DR can be estimated by removing a component attributed to vehicle motion from head motion. The following will describe in detail an effect of such a motion estimation method according to FIG. 6 through FIG. 8. Each graph shown in FIG. 6 through FIG. 8 indicates a correlation between an elapsed time and an angle of the head HD in the yaw direction yawH when the vehicle 110 travels around a cube-shaped building (see FIG. 9). The vehicle 110 travels around the building by repeatedly taking a left turn.

FIG. 6 shows a change in head angle computed by the terminal control unit 45 according to head motion detected by the head motion detection unit 11. The head angle shown in FIG. 6 is an absolute angle of the head HD with respect to a ground. Hence, a head angle changes not only when the driver DR turns the head HD by looking aside, but also when the driver DR is steering the vehicle 110 to the left.

FIG. 7 shows a change in turning angle of the vehicle 110 computed by the terminal control unit 45 according to vehicle motion detected by the mobile body motion detection unit 41. A head angle shown in FIG. 7 is an absolute angle of the vehicle 110 with respect to the ground. Hence, a turning angle changes substantially only when the driver DR is steering the vehicle 110 to the left.

FIG. 8 shows a result when a value of the turning angle shown in FIG. 7 is subtracted from a value of the head angle shown in FIG. 6. A head angle shown in FIG. 8 is a relative angle of the head HD with respect to the vehicle 110, and takes a value specifying a right-left face orientation of the driver DR with respect to a moving direction of the vehicle 110. Relative angles of the head HD in the pitch direction pitH and the roll direction rolH with respect to the vehicle 110 can be also computed by computation process same as computation process in the yaw direction yawH.

The above has described the first embodiment, in which a component attributed to vehicle motion is removed from head motion according to a difference between head motion obtained from the head motion detection unit 11 and vehicle motion obtained from the mobile body motion detection unit 41. Consequently, a change in relative angle of the head HD with respect to the vehicle 110, that is, a movement of the head HD made by the driver DR is extracted by correcting an absolute angle computed from the head motion. Hence, even in a circumstance where the driver DR wearing the head motion detection unit 11 is traveling on the vehicle 110, a movement of the head HD made by the driver DR can be detected with high accuracy.

In the first embodiment, measurement data of the acceleration sensors 12 and 42 and the gyro sensors 13 and 43 are available to the motion detection units 11 and 41 when estimating a movement of the head HD. The terminal control unit 45 is thus capable of maintaining high accuracy for a detection of a head movement by correcting the measurement data of the gyro sensors 13 and 43 with the measurement data of the acceleration sensors 12 and 42, respectively.

In addition, an acting direction of gravitational acceleration can be specified in each of the motion detection units 11 and 41 by using the sensors described above. Hence, axial alignment between three axes and corresponding three axes necessary to estimate a movement of the head HD can be performed with high accuracy. Consequently, a movement of the head HD made by the driver DR can be detected at a higher degree of accuracy.

For example, when the vehicle 110 is not moving or slowing down, the driver DR is likely to turn the head HD fully from side to side to look both sides. When the vehicle 110 is moving backward, the driver DR is likely to turn the head HD to a direction other than a usual direction to check a rearview monitor or a rear side of the vehicle 110. In such a circumstance, face orientation information is less necessary for use in the application. Hence, in the first embodiment, motion states of the vehicle 110 when not moving, slowing down, and moving backward as described above, are set as the interruption condition, and the terminal control unit 45 interrupts an estimation of a movement of the head HD when the interruption condition is satisfied. The portable terminal 40a is thus capable of reducing power consumed by estimating a movement of the head HD.

In the first embodiment, the head motion detection unit 11 is worn on the head HD of the driver DR. Hence, the head motion detection unit 11 moves integrally with the head HD of the driver DR. This configuration makes it easier to accurately understand motion of the head HD. The terminal control unit 45 is thus capable of estimating a head movement at a further higher degree of accuracy by subtracting a component attributed to vehicle motion from accurate motion information of the head HD.

In the first embodiment, the head motion detection unit 11 is attached to the eyeglasses 10a. Hence, the driver DR can wear the head motion detection unit 11 as a measurement instrument on the head HD with an improved convenience. In addition, the eyeglasses 10a worn on the head HD of the driver DR hardly slips off the head HD. Accordingly, the head motion detection unit 11 attached to the eyeglasses 10a is capable of detecting head motion accurately. The terminal control unit 45 is thus capable of estimating a head movement made by the driver DR at a further higher degree of accuracy.

In the first embodiment, the portable terminal 40a daily used by the driver DR is brought into the vehicle 110 and functions as the vehicle-mounted device 40. By using the portable terminal 40a, it becomes easy for the driver DR to launch an application and the driver DR feels less uncomfortable when using the application. The driver DR is thus made to use an abnormality warning application in a reliable manner, which prevents a quality degradation of a safety confirming action by the driver DR. In addition, because a motion sensor included in the portable terminal 40a is available as the mobile body motion detection unit 41, it is no longer necessary to add a large number of sensors to the vehicle 110.

In the first embodiment, the portable terminal 40a is attached to the instrument panel of the vehicle 110 by the holder 60. Hence, the mobile body motion detection unit 41 which is restricted from moving relatively with respect to the vehicle 110 is capable of detecting motion of the vehicle 110 accurately. The terminal control unit 45 is thus capable of estimating a head movement at a further higher degree of accuracy by exactly subtracting a component attributed to the vehicle motion from the head motion.

In the first embodiment, the main processor 45a in the portable terminal 40a performs the computation process to specify a face orientation. With the system configuration as above, computation performance required for the wearable device 10 is not high and a capacity of the battery 49 included in the wearable device 10 can be reduced. Consequently, head motion can be detected over a long period of time while the wearable device 10 having reduced weight and compact size is worn on the driver DR more steadily.

In the first embodiment, a movement state relating to a movement estimation result of the head HD can be displayed on the display 50. The display 50 is capable of displaying an image urging the driver DR to confirm surroundings of the vehicle 110 and activating an alarm when the vehicle 110 passes a near miss point. An alert may be displayed on the display 50 when a confirmation by the driver DR is inadequate, in which case the motion estimation system 100 is capable of making a contribution to an improvement of a driving skill of the driver DR. The motion estimation system 100 is also capable of monitoring a movement of the head HD and giving a warning when the driver DR confirms the surroundings of the vehicle 110 less frequently than necessary during a predetermined duration.

In the first embodiment, the acceleration sensor 12 corresponds to “a head acceleration sensor” and the gyro sensor 13 to “a head gyro sensor”. The acceleration sensor 42 corresponds to “a mobile body acceleration sensor” and the gyro sensor 43 to “a mobile body gyro sensor”. The terminal control unit 45 corresponds to “a movement estimation unit”, the main processor 45a to “a processor”, the display 50 to “an information display unit”, the vehicle 110 to “a mobile body”, and the driver DR to “an occupant”. Also, process executed in S102 corresponds to “a head motion obtaining step”, process executed in S103 corresponds to “a mobile body motion obtaining step”, and process executed in S105 to “a movement estimating step”.

### Second Embodiment

A second embodiment of the present disclosure shown in FIG. 10 and FIG. 11 is a modification of the first embodiment above. A motion estimation system 200 of the second embodiment includes a wearable device 210, a vehicle-mounted ECU 140 as a vehicle-mounted device, and a portable terminal 240a.

The wearable device 210 is a badge-shaped motion sensor device, and includes a detection circuit 220 attached to a badge 210a. The wearable device 210 is attachable to, for example, a side face of a hat a driver DR is wearing (see FIG. 1) by an attachment tool, such as a pin or a clip. The detection circuit 220 in the wearable device 210 includes a head motion detection unit 211 in addition to a communication control unit 17, an operation unit 18, and a battery 19, which are components substantially same as counterparts in the first embodiment above.

The head motion detection unit 211 has a gyro sensor 13. Meanwhile, a detection unit corresponding to the acceleration sensor 12 of the first embodiment above (see FIG. 3) is omitted from the head motion detection unit 211. The head motion detection unit 211 outputs angular velocity data about respective axes measured by the gyro sensor 13 to the communication control unit 17.

The vehicle-mounted ECU (Electronic Control Unit) 140 is a computation device equipped to a vehicle 110 (see FIG. 2) to control a vehicle posture. The vehicle-mounted ECU 140 has a mobile body motion detection unit 241, a vehicle signal obtaining unit 141 together with a control unit, such as a micro-computer.

The mobile body motion detection unit 241 is a sensor and is configured to detect motion of the vehicle 110. The mobile body motion detection unit 241 includes at least a gyro sensor 43. The mobile body motion detection unit 241 outputs angular velocity data of respective three axes measured by the gyro sensor 43 to the portable terminal 240a.

The vehicle signal obtaining unit 141 is connected to a communication bus 142 constituting an intra-vehicle network, such as CAN (Controller Area Network, registered trademark). The vehicle signal obtaining unit 141 is capable of obtaining a vehicle speed pulse outputted to the communication bus 142. A vehicle speed pulse is a signal indicating a traveling speed of the vehicle 110. The vehicle-mounted ECU is capable of calculating a present traveling speed from a vehicle speed pulse obtained by the vehicle speed obtaining unit 141 and outputting the calculated traveling speed to a wired communication unit 247b of the portable terminal 240a as vehicle speed data.

The portable terminal 240a includes a wireless communication unit 247a, the wired communication unit 247b, and a power feed unit 249 in addition to a terminal control unit 45 and a memory 46. The terminal control unit 45 and the memory 46 are substantially same as counterparts in the first embodiment above. The wireless communication unit 247a corresponds to the communication unit 47 of the first embodiment above (see FIG. 3) and transmits information to and receives information from the communication control unit 17 by wireless communication.

The wired communication unit 247b is connected to the vehicle-mounted ECU 140. The wired communication unit 247b outputs angular velocity data and vehicle speed data acquired from the vehicle-mounted ECU 140 to the main processor 45a. The power feed unit 249 is connected to a vehicle-mounted power supply 120. The power feed unit 249 supplies power from the vehicle-mounted power supply 120 to respective elements in the portable terminal 240a. Alternatively, the wired communication unit 247b may be directly connected to the communication bus 142. When configured in such a manner, the portable terminal 240a is capable of obtaining a traveling speed of the vehicle 110 without depending on the vehicle speed data outputted from the vehicle-mounted ECU 140.

By performing process corresponding to S105 of the first embodiment above (see FIG. 5), the terminal control unit 45 obtains angular velocities about a pitch direction pitH, a yaw direction yawH, and a roll direction rolH (see FIG. 1) according to measurement data of the gyro sensor 13. The measurement data of the gyro sensor 13 is acquired by wireless communication. The terminal control unit 45 also obtains angular velocities about respective directions relating to vehicle motion according to measurement data of the gyro sensor 43. The measurement data of the gyro sensor 43 is acquired by wired communication. The terminal control unit 45 calculates differences of the angular velocities between head motion and vehicle motion, and calculates an angle of a head HD by integrating the differences. The terminal control unit 45 is thus capable of estimating a relative movement of the head HD with respect to the vehicle 110. Also, by performing process corresponding to S108 of the first embodiment above (see FIG. 5), the terminal control unit 45 becomes capable of estimating a motion state of the vehicle 110 while the vehicle is not moving, slowing down, or moving backward.

In the second embodiment described above, too, a head movement made by the driver can be estimated by the computation process of the terminal control unit 45 as in the first embodiment above. Hence, even in a circumstance where the driver DR wearing the badge 210a is travelling on the vehicle 110 (see FIG. 2), a movement of the head HD can be detected with high accuracy.

In the second embodiment, a sensor in the vehicle-mounted ECU 140 equipped to the vehicle 110 (see FIG. 2) is used as the mobile body motion detection unit 241. The vehicle-mounted ECU 140 is attached to the vehicle 110 in a reliable manner. Hence, the gyro sensor 43 is capable of measuring motion of the vehicle 110 accurately and outputting accurate measurement data to the portable terminal 240a. Consequently, a head movement can be estimated at a higher degree of accuracy.

By supplying power from the vehicle-mounted power supply 120 to the portable terminal 240a as in the second embodiment, an application freeze occurring due to a low state of charge of the battery 49 in the portable terminal 240a can be prevented. Hence, an estimation of a head movement can be continued in a reliable manner while the driver DR is driving.

In the second embodiment, vehicle speed data is used to estimate a motion state of the vehicle 110. Hence, estimation accuracy for a motion state can be maintained at a high degree. Consequently, computation process can be interrupted at appropriate timing. In the second embodiment, the vehicle-mounted ECU 140 corresponds to “a vehicle-mounted device”.

### Third Embodiment

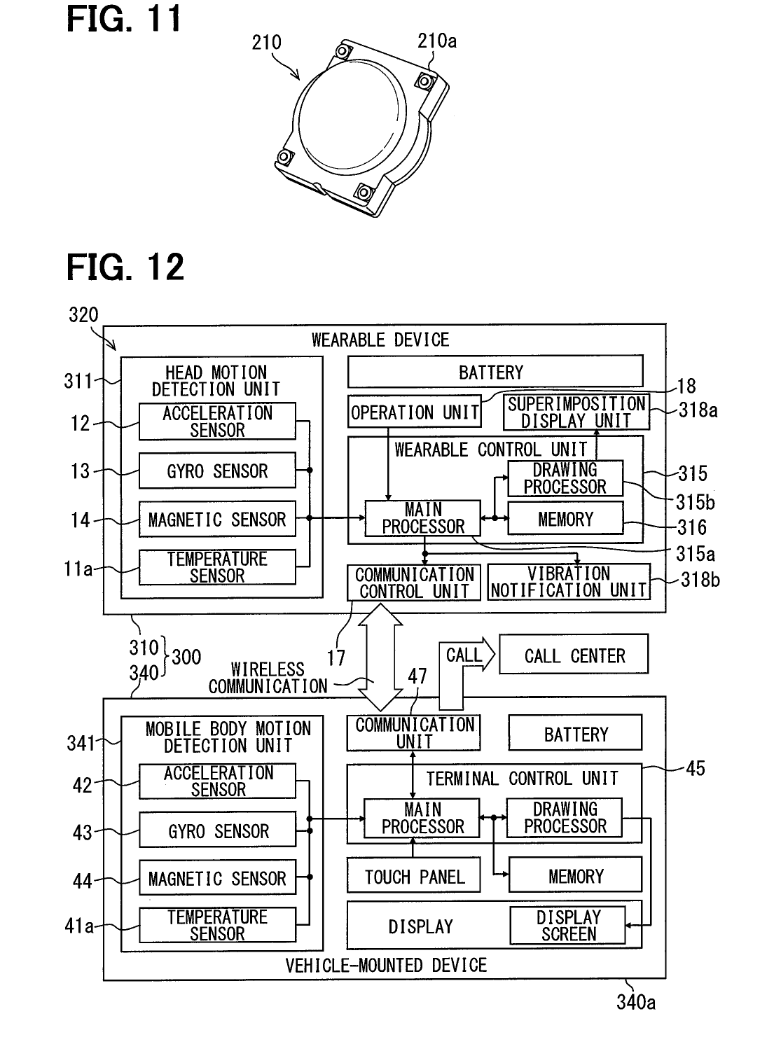

A third embodiment of the present disclosure shown in FIG. 12 is another modification of the first embodiment above. A motion estimation system 300 of the third embodiment includes a wearable device 310 and a portable terminal 340a as a vehicle-mounted device 340. In the motion estimation system 300, signal processing to estimate a face orientation is performed by the wearable device 310.

A detection circuit 320 provided to the wearable device 310 has a head motion detection unit 311, a wearable control unit 315, a superimposition display unit 318a, and a vibration notification unit 318b in addition to a communication control unit 17 and an operation unit 18, both of which are substantially same as counterparts in the first embodiment above.

The head motion detection unit 311 includes a magnetic sensor 14 and a temperature sensor 11a in addition to an acceleration sensor 12 and a gyro sensor 13. The magnetic sensor 14 is configured to detect a magnetic field acting on the head motion detection unit 311, such as a magnetic field released from earth magnetism and vehicle-mounted devices. The magnetic sensor 14 is capable of measuring magnitude of magnetic fields in respective axial directions along an Xw axis, a Yw axis, and a Zw axis (see FIG. 1). When a posture of the head motion detection unit 311 changes with a movement of the head HD, a magnetic orientation acting on the magnetic sensor 14 changes, too. The magnetic sensor 14 outputs magnetic data of the respective three axes, which increases and decreases with a change in posture of the head motion detection unit 311, to the wearable control unit 315.

The temperature sensor 11a is configured to detect a temperature of the head motion detection unit 311. The temperature sensor 11a outputs measured temperature data to the wearable control unit 315. The measured temperature data of the temperature sensor 11a is used to correct an offset of a zero-point position of the gyro sensor 13 occurring in response to a temperature change.

The wearable control unit 315 is mainly formed of a microcomputer having a main processor 315a, a drawing processor 315b, a RAM, a flash memory 316, an input-output interface, and so on. The wearable control unit 315 acquires measurement data of head motion from the head motion detection unit 311. The wearable control unit 315 uses the communication control unit 17 as a receiver and acquires measurement data of vehicle motion of the mobile object motion detection unit 341 from the portable terminal 340a by wireless communication. As with the terminal control unit 45 of the first embodiment above (see FIG. 2), the wearable control unit 315 is capable of computing a face orientation of a driver DR (see FIG. 1) by executing a program read out from the flash memory 316.

During the computation process, the wearable control unit 315 outputs a command signal instructing the portable terminal 340a to start a detection of vehicle motion from the communication control unit 17 to the portable terminal 340a as process corresponding to S101 of the first embodiment above (see FIG. 5). In response to the command signal from the wearable device 310, a terminal control unit 45 in the portable terminal 340a starts a detection of vehicle motion by the mobile body motion detection unit 341 and a transmission of measurement data by a communication unit 47.

The superimposition display unit 318a is capable of displaying an image superimposed on a field of view of the driver DR by projecting various images onto a half mirror or the like provided ahead of lenses of eyeglasses 10a (see FIG. 4). The superimposition display unit 318a is connected to the wearable control unit 315 and a display by the superimposition display unit 318a is controlled by the wearable control unit 315. The superimposition display unit 318a is capable of displaying a warning image superimposed on a field of view of the driver DR when a quality of a safety confirming action degrades. The superimposition display unit 318a is further capable of urging the driver DR to confirm surroundings of the vehicle 110 by displaying a superimposed image when the vehicle 110 passes a near miss point.

The vibration notification unit 318b is a vibration motor provided to the eyeglasses 10a (see FIG. 4). The vibration notification unit 318b is capable of providing a notification to the driver DR wearing the wearable device by vibrating a vibrator attached to a rotation shaft of the vibration motor. The vibration notification unit 318b is connected to the wearable control unit 315 and an operation of the vibration notification unit 318b is controlled by the wearable control unit 315. The vibration notification unit 318b is capable of bringing the driver DR who becomes distractive by, for example, looking aside for a considerable time back to a normal driving state by calling an attention with vibration.

The portable terminal 340a transmits vehicle motion detected by the mobile body motion detection unit 341 from the communication unit 47 to the wearable device 310. The mobile body motion detection unit 341 in the portable terminal 340a is provided with a magnetic sensor 44 and a temperature sensor 41a in addition to an acceleration sensor 42 and a gyro sensor 43.

The magnetic sensor 44 measures a magnetic field acting on the mobile body motion detection unit 341. When a posture of the vehicle 110 changes in response to an operation by the driver DR or from any other cause, a magnetic orientation acting on the magnetic sensor 44 changes, too. The magnetic sensor 44 outputs magnetic data of respective three axes, which increases and decreases with a change in posture of the mobile body motion detection unit 341, to the terminal control unit 45.

The temperature sensor 41a detects a temperature of the mobile body motion detection unit 341 and outputs measured temperature data to the terminal control unit 45. The temperature data of the temperature sensor 41a is used to correct an offset of a zero-point position of the gyro sensor 43.

Even when the computation process to estimate a face orientation is performed by the wearable device 310 as in the third embodiment described above, a head movement can be estimated as in the first embodiment above. In addition, in the third embodiment, not only the measurement data of the gyro sensors 13 and 43, but also measurement data of the magnetic sensor 14 and 44 can be used for axial alignment between the two motion detection units 311 and 341 (see S104 of FIG. 5) and a calculation of an angle of the head HD (see S105 of FIG. 5). Owing to a correction using the magnetic sensors 14 and 44 as above, a head movement can be detected at a further higher degree of accuracy.

In the third embodiment, the wearable control unit 315 corresponds to “the movement estimation unit”, the main processor 315a to “the processor”, the magnetic sensor 14 to “a head magnetic sensor”, and the magnetic sensor 44 to “a mobile body magnetic sensor”.

### Fourth Embodiment

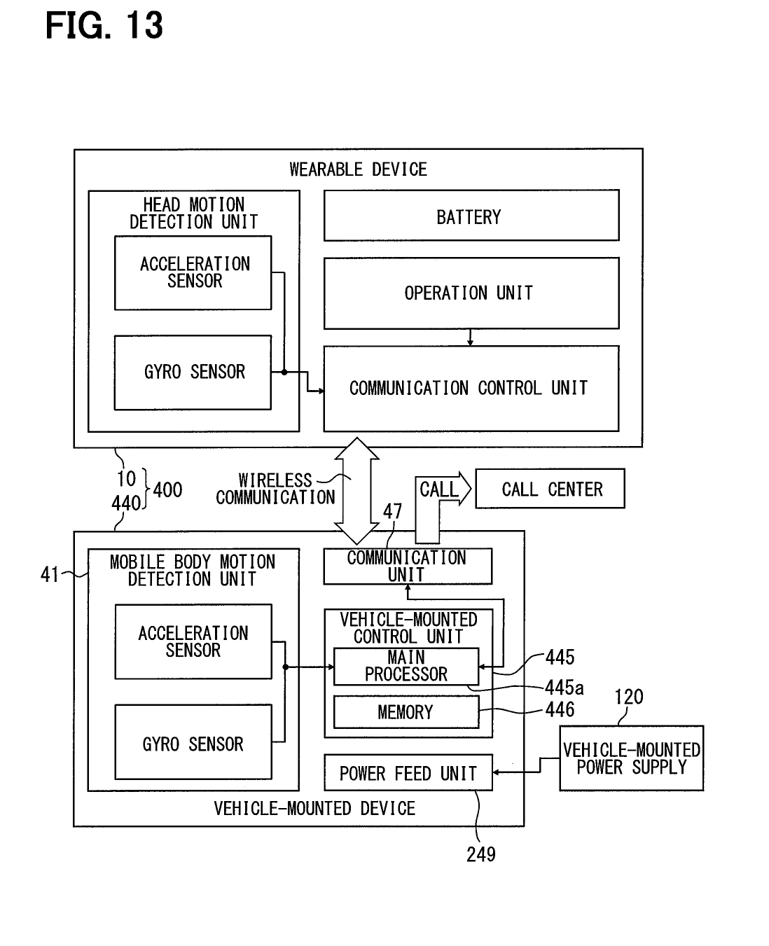

A fourth embodiment of the present disclosure shown in FIG. 13 is another modification of the first embodiment above. A motion estimation system 400 of the fourth embodiment includes a wearable device 10 and a vehicle-mounted device 440. The vehicle-mounted device 440 is a control unit mounted to a vehicle 110 (see FIG. 2). The vehicle-mounted device 440 is fixed to a frame of the vehicle 110 or the like by a fastening member. The vehicle-mounted device 440 includes a mobile body motion detection unit 41, a communication unit 47, and a vehicle-mounted control unit 445. The vehicle-mounted device 440 operates on power supplied from a vehicle-mounted power supply 120 to a power feed unit 249.

The vehicle-mounted control unit 445 is mainly formed of a microcomputer having a main processor 445a, a RAM, a memory 446, an input-output interface, and so on. The main processor 445a is substantially same as the main processor 45a of the first embodiment above (see FIG. 3) and performs computation process to estimate a face orientation by executing a program read out from the memory 446.

Even when a portable device is not used and the vehicle-mounted device 440 estimating a face orientation is provided instead of the portable device as in the fourth embodiment, a head movement can be estimated as in the first embodiment above. In the fourth embodiment, the vehicle-mounted control unit 445 corresponds to “the movement estimation unit” and the main processor 445a to “the processor”.

### Other Embodiments

While the disclosure has been described with reference to above-described embodiments thereof, it is to be understood that the disclosure is not limited to the above embodiments and constructions. The disclosure is intended to cover various modification and equivalent arrangements. In addition, the various combinations and configurations, which are preferred, other combinations and configurations, including more, less or only a single element, are also within the spirit and scope of the disclosure.

In the embodiments above, signal processing relating to the face orientation estimation is performed entirely by the processor in either the wearable device or in the vehicle-mounted device. Alternatively, respective process steps for the face orientation estimation may be allocated to and performed by the both of the wearable device and the vehicle-mounted device.

In a configuration using no portable terminal as in the fourth embodiment above, a vehicle-mounted control unit may be a processing device used exclusively to estimate a face orientation. Alternatively, a navigation device, an HCU (HMI Control Unit), or the like may also function as a control unit for an estimation of a face orientation. The term, “HMI”, referred to herein stands for Human Machine Interface.

A face orientation estimation function provided by the respective processors in the embodiments as described above may be provided by hardware or software in different manner from the structure described above, or may be provided by a combination of hardware and software.

(First Modification)

In each of the embodiments described above, a gyro sensor is provided to the respective motion detection units. In a first modification of the first embodiment above, a gyro sensor is omitted from the respective motion detection units. In the first modification, an angle of the head is calculated according to measurement data of the triaxial acceleration sensors 12 and 42 provided to the respective motion detection units.

In the embodiments described above, the wearable device and the vehicle-mounted device are connected to each other by wireless communication and configured to exchange measurement information of the respective sensors. Alternatively, the wireless communication can be changed as needed. Further, the wearable device and the vehicle-mounted device may be wire-connected to each other by, for example, a flexible cable.

The respective acceleration sensors and the respective gyro sensors used in the embodiments above are preferably capacitance or piezo-resistive sensors formed by using, for example, a MEMS (Micro Electro Mechanical Systems) technique. A magnetic sensor using a magneto-resistive element which changes a resistance value depending on whether a magnetic field is present or absent, a fluxgate magnetic sensor, a magnetic sensor using a magnetic impedance element or a hall element, and so on are also adoptable in the respective motion detection units.

The embodiments above have described the wearable device in the shape of eyeglasses or a badge as examples. Alternatively, a wearing method of the wearable device on the head HD can be changed as needed. For example, the wearable device may be of an ear-hook type hooked behind ears. The wearable device may be of a hat shape formed by embedding a detection circuit in a hat. Wearable devices of such types are particularly suitable for a driver engaged in transportation industry, such as a home delivery service.

In the embodiments above, sensor provided to the head motion detection unit is same as the sensor provided to the mobile body motion detection unit. Alternatively, sensors provided to the head motion detection unit and the mobile body motion detection unit may be of different types. For example, information on a moving direction and a moving speed of a vehicle found from GPS (Global Positioning System) data may be used to correct measurement data of each sensor.

In the embodiments above, the computation process to estimate a movement by the terminal control unit or the wearable control unit is temporarily interrupted when the vehicle is not moving, slowing down, or moving backward. Owing to such interruption process, not only can power consumption be reduced in the respective control units, but also an unnecessary warning to an occupant can be prevented. Alternatively, an unnecessary warning may be prevented by merely interrupting a warning according to face orientation information when the vehicle is not moving, slowing down, or moving backward.

The motion estimation systems according to the embodiments above estimate a movement of the head by inertial sensors alone. Alternatively, the motion estimation systems may be configured to combine a head movement estimated by an inertial sensor and a head movement extracted from a camera image. When configured in such a manner, the motion estimation systems become capable of detecting an abnormal state of the driver or any other individual at a further higher degree of accuracy.

A head movement can be estimated by the motion estimation systems in a mobile body different from the mobile bodies of the embodiments above. A mobile body may include a personal vehicle, a cargo vehicle (truck), a tractor, a motorbike (two-wheel vehicle), a bus, a construction machine, an agricultural machine, a ship, an airplane, a helicopter, a train, and a streetcar. In addition, the occupant is not limited to the driver of the vehicle as in the embodiments above. Examples of the occupant include but not limited to a pilot of an air plane, a train operator, and an occupant seated in a front occupant seat of a vehicle. The occupant may further include an operator (driver) being monitored in an automatically operated vehicle.

What is claimed is:

1. A motion estimation system, comprising:
a head motion detection unit worn on an occupant in a mobile body and detecting a motion of a head of the occupant;
a mobile body motion detection unit detecting a motion of the mobile body; and
a movement estimation unit obtaining the motion of the head of the occupant and the motion of the mobile body and estimating a movement of the head of the occupant made by the occupant according to a difference between the motion of the head of the occupant and the motion of the mobile body.

2. The motion estimation system according to claim 1, wherein:
the head motion detection unit includes a head gyro sensor measuring an angular velocity of the head of the occupant;
the mobile body motion detection unit includes a mobile body gyro sensor measuring an angular velocity of the mobile body; and
the movement estimation unit uses estimates the movement of the head of the occupant with reference to measurement information of the head gyro sensor and measurement information of the mobile body gyro sensor.

3. The motion estimation system according to claim 2, wherein:
the head motion detection unit includes a head acceleration sensor measuring an acceleration of the head of the occupant;
the mobile body motion detection unit includes a mobile body acceleration sensor measuring an acceleration of the mobile body; and
the movement estimation unit estimates the movement of the head of the occupant with reference to measurement information of the head acceleration sensor and measurement information of the mobile body acceleration sensor.

4. The motion estimation system according to claim 3, wherein:
the head motion detection unit includes a head magnetic sensor measuring a magnetic field applied to the head motion detection unit;
the mobile body motion detection unit includes a mobile body magnetic sensor measuring a magnetic field applied to the mobile body motion detection unit; and
the movement estimation unit estimates the movement of the head of the occupant with reference to measurement information of the head magnetic sensor and measurement information of the mobile body magnetic sensor.

5. The motion estimation system according to claim 1, wherein:
the head motion detection unit includes a head acceleration sensor measuring an acceleration of the head of the occupant;
the mobile body motion detection unit includes a mobile body acceleration sensor measuring an acceleration of the mobile body; and
the movement estimation unit estimates the movement of the head of the occupant with reference to measurement information of the head acceleration sensor and measurement information of the mobile body acceleration sensor.

6. The motion estimation system according to claim 3, wherein:
the movement estimation unit interrupts an estimation of the movement of the head of the occupant when the motion of the mobile body according to the measurement information of the mobile body acceleration sensor satisfies an interruption condition that is preliminarily set.

7. The motion estimation system according to claim 6, wherein:
the interruption condition is satisfied at least when the mobile body is not moving, when the mobile body is moving slowly at a speed equal to or lower than a predetermined speed, or when the mobile body is moving backward.

8. The motion estimation system according to claim 1, wherein:
the head motion detection unit is worn on the head of the occupant.

9. The motion estimation system according to claim 1, wherein:
the head motion detection unit is included in eyeglasses worn on the head of the occupant.

10. The motion estimation system according to claim 1, wherein:
the mobile body motion detection unit is included in a portable terminal carried into the mobile body by the occupant.

11. The motion estimation system according to claim 1, wherein:
the mobile body motion detection unit is attached to the mobile body and restricted from moving relatively with respect to the mobile body.

12. The motion estimation system according to claim 1, wherein:
the head motion detection unit is included in a wearable device worn on the occupant;
the mobile body motion detection unit is included in a vehicle-mounted device mounted on the mobile body; and
the movement estimation unit is included in the vehicle-mounted device.

13. The motion estimation system according to claim 1, wherein:
the head motion detection unit is included in a wearable device worn on the occupant;
the mobile body motion detection unit is included in a vehicle-mounted device mounted on the mobile body; and
the movement estimation unit is included in the wearable device.

14. The motion estimation system according to claim 1, further comprising:
an information display unit displaying information indicating an operating state of the movement estimation unit.

15. A motion estimation method executed by at least one processor comprising:
obtaining a motion of a head of an occupant in a mobile body, the motion of the head of the occupant being detected by a wearable device worn on the occupant;
obtaining a motion of the mobile body detected by a vehicle-mounted device mounted on the mobile body; and
estimating a movement of the head made by the occupant according to a difference between the motion of the head of the occupant and the motion of the mobile body.

16. A wearable device employed in a motion estimation system, the wearable device comprising:
a head motion detection unit detecting a motion of a head of an occupant in a mobile body,
wherein
the motion estimation system includes:
a mobile body motion detection unit detecting a motion of the mobile body; and
a movement estimation unit obtaining the motion of the head of the occupant and the motion of the mobile body, and estimating a movement of the head made by the occupant according to a difference between the motion of the head and the motion of the mobile body, and
the wearable device is worn on the occupant.
